(19) United States
(12) Patent Application Publication
(10) Pub. No.: US 2025/0306009 A1
(43) Pub. Date: Oct. 2, 2025

(54) SYSTEM AND METHOD FOR REGULATING LIVING TISSUE CELL

(71) Applicant: The Hong Kong Polytechnic University, Hung Hom Kowloon (HK)

(72) Inventors: Puxiang LAI, Hung Hom Kowloon (HK); Tianting ZHONG, Hung Hom Kowloon (HK); Chi Man WOO, Hung Hom Kowloon (HK)

(21) Appl. No.: 18/760,667

(22) Filed: Jul. 1, 2024

(30) Foreign Application Priority Data
Mar. 29, 2024 (CN) 202410381387.3

(51) Int. Cl. G01N 33/50; C12M 1/00; C12M 1/34

(52) U.S. Cl. CPC G01N 33/5023; C12M 41/06; C12M 41/46

(57) ABSTRACT

A system and a method for regulating a living tissue cell are provided. The system includes: a laser source module, a modulation module, an optical fiber module, an imaging module, and a control module. The modulation module is configured for modulating the received first laser based on a preset modulation strategy to determine a regulation light and modulating the received second laser based on the preset modulation strategy to determine an imaging light, wherein the preset modulation strategy refers to a strategy for wavefront modulation of the first laser and the second laser. The regulation light or the imaging light is output to the deep area of living tissue through the multimode optical fiber in the optical fiber module.

### CROSS-REFERENCE TO RELATED APPLICATIONS

This application claims priority to Chinese Patent Application No. 202410381387.3 filed on Mar. 29, 2024, the contents of which are incorporated herein by reference in their entirety.

### TECHNICAL FIELD

The present application relates to the field of biomedical technology, and in particular to a system and a method for regulating a living tissue cell.

### BACKGROUND

Optogenetics involves expressing photosensitive proteins in specific cells (such as neurons, etc.), and then regulating activities of such specific cells through light of different wavelengths.

In the field of biomedicine, the living tissue corresponding to specific cells can scatter and absorb light, which affects the propagation of light and limits the penetration depth of light within the tissue. In such case, how to realize in vivo optogenetic control of deep regions of living tissue is an urgent problem to be solved.

### SUMMARY

The present application provides a system and a method for regulating a living tissue cell, which can realize optogenetic control of deep areas in living tissue.

In a first aspect, a system for regulating a living tissue cell is provided. The system comprises: a laser source module, a modulation module, an optical fiber module, an imaging module, and a control module. The control module is in communication connection with the imaging module.

The laser source module is configured for generating a first laser and a second laser. The modulation module is configured for modulating the received first laser based on a preset modulation strategy to determine a regulation light, and modulating the received second laser based on the preset modulation strategy to determine an imaging light, where the preset modulation strategy is a strategy for wavefront modulation of the first laser and the second laser. The optical fiber module is configured for outputting the regulation light or the imaging light. The imaging module is configured for receiving a fluorescence signal in the target area through the optical fiber module and determining a detection image corresponding to the target area based on the fluorescence signal. The control module is configured for determining a target regulation area where the target cell is located based on the detection image, so that the modulated light can be adopted to regulate target cell in the target regulation area.

It should be understood that the first laser is configured for regulating a target cell based on optogenetics, and the second laser is configured for imaging the target area. Similarly, after modulation, the regulation light is further configured for regulating the target cell, and the imaging light is used to image the target area. The target area refers to the area to be regulated in the living tissue where the target cell is located. Therefore, the target regulation area where the target cell is located belongs to the area to be regulated.

In some embodiments, the area to be regulated in the living tissue may be located in a superficial area of the living tissue, or may be located a deep area of the living tissue. For example, for the living tissue of the brain, the area to be regulated may be deep brain regions.

That is to say, the regulation light or imaging light obtained from modulation of the first laser or the second laser by the modulation module can be output by the optical fiber module, and the detection image corresponding to the target area is determined by the imaging module, thereby realizing the visualization of the target area and facilitating the positioning of the target cell in the target area, and further, by determining the target regulation area where the target cell is located in the detection image, the target cell can be precisely positioned to facilitate precise control of the target cell. It should be understood that the target cell may be a single cell.

In a possible implementation, the modulation module comprises a light regulation unit and a modulation unit. The light regulation unit is configured for expanding or scaling a process light passing through the modulation module. The process light at least comprises the first laser and the second laser. The modulation unit is configured for adjusting a phase and an intensity of the process light based on the preset modulation strategy.

In some embodiments, the adjustment of the phase and the intensity of the process light can be accomplished through the same optical element.

The modulation module realizes wavefront modulation through the preset modulation strategy, and performs high-speed and diffraction-limited focusing through the optical fibers to achieve precise positioning of target cells.

In such condition, in a possible implementation, the light regulation unit comprises: a first lens assembly and a second lens assembly. The modulation unit comprises a holographic modulation assembly. The first lens assembly is configured for expanding the first laser and the second laser. The holographic modulation assembly is configured for adjusting a phase and an intensity of the first laser and the second laser after being expanded based on a preset modulation strategy to obtain a first output light and a second output light. The second lens assembly is configured for scaling the first output light and the second output light and determining the regulation light and the imaging light.

In some embodiments, the preset modulation strategy realizes the adjustment of the expanded first laser and the expanded second laser through the preset hologram. The holographic modulation assembly realizes wavefront adjustment of the first laser and the second laser by displaying a preset hologram.

The above-mentioned combination of the first lens assembly, the holographic modulation assembly, and the second lens assembly realizes the wavefront adjustment of the first laser and the second laser, in which, the first laser and the second laser pass through the first lens assembly, the holographic modulation assembly, and the second lens assembly, respectively, so as to determine the regulation light configured for regulating the target cell and the imaging light configured for imaging the target area, so that the regulation light and the imaging light can be output through the same optical fiber module, thereby making the system can not only image the target area and precisely locate the target cell, but also control the target cell.

In another possible implementation, the light regulation unit further comprises a screening assembly. The screening assembly is configured for screening a first target order of the first output light from different diffraction orders corresponding to the first output light and a first target order of the second output light from different diffraction orders corresponding to the second output light. The regulation light is a light whose diffraction order is the first target order in the first output light, and the imaging light is a light whose diffraction order is the first target order in the second output light.

It should be understood that the wavelengths of the first laser and the second laser are different, and by adjusting output light fields of the expanded first laser and the expanded second laser based on the preset hologram, the determined first output light and second output light are also different. Based on the preset hologram, both the first output light and the second output light can have corresponding target orders that can pass through the screening assembly, so that the determined regulation light and imaging light can be output through the same optical fiber module, thereby simplifying the part inserted into the living tissue.

In a possible implementation, the laser source module comprises a beam splitting unit. The beam splitting unit is configured for dividing the second laser into a second signal beam and a reference beam. The second signal beam is configured for imaging the target area. The reference beam is configured for calibrating the second signal beam.

In a possible implementation, the system further comprises a calibration module, and the calibration module comprises a reference unit and a recording unit. The reference unit is configured for converging the reference beam to the recording unit. The recording unit is configured for: acquiring a combined beam, and determining a first transmission matrix based on interference between different beams in the combined beam. The combined beam comprises the reference beam and an object beam, and the object beam refers to the regulation light or the imaging light which is irradiated on the target area.

In order to make the measurement of the first transmission matrix more precise, the second laser is divided by a beam splitting unit into the second signal beam and the reference beam. Based on the interference between the reference beam and the light output from the multimode optical fiber, the first transmission matrix can be more precise.

In a possible implementation, the reference unit further comprises a shutter assembly. The shutter assembly is configured for controlling the reference beam to converge to the recording unit or blocking the reference beam from converging to the recording unit. In a case where the shutter assembly blocks the reference beam from converging to the recording unit, the recording unit is configured for acquiring the object beam and determining a second transmission matrix based on the object beam.

It should be understood that the reference beam is used for calibration of the device that emits the second laser, and for calibration of the device that emits the first laser, the reference beam is not required.

In a possible implementation, the control module is further in communication connection with the modulation module; the control module is further configured for determining a preset modulation strategy. The preset modulation strategy comprises a first modulation strategy and a second modulation strategy. The first modulation strategy is configured for wavefront modulation of the first laser, and the second modulation strategy is configured for wavefront modulation of the second laser.

It should be understood that the wavelengths of the first laser and the second laser are different. In order to allow both the first laser and the second laser to pass through the optical fiber module without interfering with each other, the preset modulation strategy is adopted, and the first laser and the second laser respectively have different first and second modulation strategies. When the first laser is performed with wavefront modulation, the second laser cannot be output to the optical fiber module. When the second laser is performed with wavefront modulation, the first laser cannot be output to the optical fiber module.

In a possible implementation, the laser source module comprises: a first light regulation unit, a second light regulation unit, and a combining unit. The first light regulation unit is configured for controlling an intensity of the first laser. The second light regulation unit is configured for controlling an intensity of the second laser. The combining unit is configured for combining the first laser and the second laser into one channel of light, wherein the first laser passes through the combining unit, and the second laser is reflected by the combining unit.

The combining unit is adopted to combine the first laser and the second laser into one channel of light, which facilitates the modulation of the first laser or the second laser through the modulation module, and further facilitates the integration of the regulation light after modulation and the imaging light after modulation into a single optical fiber, such that the part of the system that configured to be inserted into the living tissue can be simplified, so as to reduce damage to living tissue.

In a second aspect, a method for regulating a living tissue cell is provided, which is applied to the system for regulating the living tissue cell according to the first aspect. The method comprises: receiving a first laser and a second laser, in which, the first laser is configured for regulating a target cell based on optogenetics, and the second laser is configured for imaging a target area, and the target area refers to an area to be regulated in a living tissue where the target cell is located; modulating the received first laser based on a preset modulation strategy to obtain a regulation light after modulation, and modulating the received second laser based on the preset modulation strategy to obtain an imaging light after modulation, in which, the preset modulation strategy refers to a strategy for wavefront modulation of the first laser and the second laser; receiving a fluorescence signal in the target area, and determining a detection image corresponding to the target area based on the fluorescence signal, in which, the fluorescence signal is a light emitted by the target area after being illuminated by the imaging light; determining a target regulation area where the target cell is located based on the detection image, in which, the target regulation area belongs to the area to be regulated; and regulating the target cell based on the regulation light in the target regulation area.

In a third aspect, a computer-readable storage medium is provided. The computer-readable storage medium stores computer program codes, which, when being executed, cause the method for regulating the living tissue cell in the second aspect to be performed.

In a fourth aspect, a computer program product is provided. The computer program product comprises computer program codes, which, when being executed, cause the method for regulating the living tissue cell in the second aspect to be performed.

It can be understood that the beneficial effects of the above-mentioned second aspect to the fourth aspect can be referred to the relevant description in the above first aspect, and will not be repeated again here.

Compared with the prior art, beneficial effects of the embodiments of the present application are summarized as follows:

Embodiments of the present application provide a system and method for regulating a living tissue cell. The system comprises a laser source module, a modulation module, an optical fiber module, an imaging module, and a control module. The laser source module is configured for generating a first laser and a second laser, the first laser is configured for regulating a target cell based on optogenetics, and the second laser is configured for imaging a target area, which refers to an area to be regulated in a living tissue where the target cell is located. In this way, the system can achieve the imaging of living tissues and the regulation (such as stimulation, inhibition, intercellular communication, etc.) of target cells in living tissues. The modulation module is configured for modulating the received first signal based on a preset modulation strategy to determine the regulation light and modulating the received second laser based on the preset modulation strategy to determine the imaging light. The regulation light is configured for controlling the target cells, and the imaging light is configured for imaging the target area. By using the same modulation module, different lasers are modulated, two beams are output without interfering with each other, and the flexible switching between imaging of the target area and the regulation of the target cells are enhanced, thereby realizing the coexistence of two kinds of lights. Further, the optical fiber module is configured for outputting a regulation light or an imaging light, in order to achieve the positioning of target cells, the imaging module is configured for receiving, via the optical fiber module, a fluorescence signal emitted by the target area after being illuminated by the imaging light, and determining the detection image corresponding to the target area based on the fluorescence signal. The control module is configured for determining a target regulation area where the target cell is located based on the detection image, so that the target cells can be precisely regulated in the target regulation area via the regulation light.

### BRIEF DESCRIPTION OF THE DRAWINGS

In order to more clearly illustrate the technical solutions in embodiments of the present application, the drawings needed to be used in the embodiments or description of the prior art will be briefly introduced below. Obviously, the drawings in the following description are only for the purpose of the present application. For some embodiments, for those of ordinary skill in the art, other drawings can be obtained based on these drawings without creative efforts.

FIG. 1 is a schematic structural diagram of a system for regulating a living tissue cell provided by an embodiment of the present application;

FIG. 2 is a schematic structural diagram of a system for regulating a living tissue cell provided by an embodiment of the present application;

FIG. 3 is a schematic structural diagram of a system for regulating a living tissue cell provided by an embodiment of the present application;

FIG. 4 is a schematic structural diagram of a system for regulating a living tissue cell provided by an embodiment of the present application;

FIG. 5 is a schematic structural diagram of a system for regulating a living tissue cell provided by an embodiment of the present application;

FIG. 6 is a comparison diagram showing effects of wavefront shaping provided by the embodiment of the present application;

FIG. 7 is imaging results of a system for regulating a living tissue cell provided by an embodiment of the present application;

FIG. 8 is regulation results of a system for regulating a living tissue cell provided by an embodiment of the present application; and

FIG. 9 is a schematic flow chart of a method for regulating a living tissue cell provided by an embodiment of the present application.

### DETAILED DESCRIPTION OF THE EMBODIMENTS

In the following description, for the purpose of explanation rather than limitation, specific details such as specific system structures and technologies are provided to provide a thorough understanding of the embodiments of the present application. However, it will be apparent to those skilled in the art that the present application may be practiced in other embodiments without these specific details. In other instances, detailed descriptions of well-known systems, devices, circuits, and methods are omitted so as not to obscure the description of the present application with unnecessary detail.

It will be understood that, when used in this specification and the appended claims, the term “comprising” indicates the presence of the described features, integers, steps, operations, elements, and/or components but does not exclude presence or addition of one or more of other features, integers, steps, operations, elements, components and/or a combination thereof.

Optogenetics involves expressing photosensitive proteins in specific cells (such as neurons, cardiomyocytes, liver cells, immune cells, etc.), and then regulating activities of such specific cells through light of different wavelengths.

The photosensitive proteins refer to a type of proteins that can produce physiological reactions in response to light signals, and can be divided into excitatory photosensitive proteins and inhibitory photosensitive proteins according to the electrophysiological functions produced by the light stimulation. For example, channelrhodopsins and halorhodopsins can be introduced into specific cells in the brain or other tissues using genetic methods. After being expressed in specific cells, the photosensitive proteins can be activated or inhibited by lights of specific wavelengths, and in turn, can manipulate the activities of these specific cells.

In the field of biomedicine, the penetration depth of light into living tissues is limited. That is to say, the living tissue corresponding to specific cells can scatter and absorb light, which affects the propagation of light and limits the penetration depth of light within the tissue. The scattering and absorption of light by living tissue depends on the thickness of living tissue. In the deep areas of the living tissue, light will be scattered out very quickly, thereby reducing the in-situ photon flux, resulting in the loss of spatial information, and making the optogenetics unable to be spatially precise and/or deeply organized.

For example, for the living tissues of the brain, the deep areas comprise: thalamus, hypothalamus, brainstem, cerebellum, etc. These areas are interconnected and work cooperatively with other parts of the brain to maintain normal physiological functions and behavioral performance of the body. Therefore, the regulation of the deep areas of living tissue is very important.

In this case, double-photon excitation is proposed. The double-photon excitation includes absorbing the energy of two photons at the same time to achieve an excited state and generate an excitation event, thereby reducing the scattering and absorption of light in living tissues and therefore locally activating or inhibiting photosensitive proteins in deeper areas of the living tissues.

The probability of the above excitation event is proportional to a square of a light intensity. Therefore, the double-photon excitation only occurs at a focus position of a laser beam, and the light intensity at the focus position is the highest. Although the double-photon excitation has better penetration ability into living tissue, in deeper living tissues, the signal will be attenuated as the depth increases, making the double-photon excitation has limited regulation effect of the specific cells in the area to be regulated in the living tissue.

It should also be understood that although the double-photon excitation reduces light scattering, high-power light may still cause a certain degree of optical damage to cells or tissues, and the double-photon excitation requires a high-intensity laser device and corresponding special devices. Such professional devices are imposed with high operational performance requirements, making the entire system difficult to operate.

In order to solve the above problems, embodiments of the present application provide a system and a method for regulating a living tissue cell. The system comprises a laser source module, a modulation module, an optical fiber module, an imaging module, and a control module. The laser source module is configured for generating a first laser and a second laser, the first laser is configured for regulating a target cell based on optogenetics, and the second laser is configured for imaging a target area, which refers to an area to be regulated in a living tissue where the target cell is located. In this way, the system can achieve the imaging of living tissues and the regulation (such as stimulation, inhibition, intercellular communication, etc.) of target cells in living tissues. The modulation module is configured for modulating the received first signal based on a preset modulation strategy to determine the regulation light and modulating the received second laser based on the preset modulation strategy to determine the imaging light. The regulation light is configured for controlling the target cells, and the imaging light is configured for imaging the target area. By using the same modulation module, different lasers are modulated, two beams are output without interfering with each other, and the flexible switching between imaging of the target area and the regulation of the target cells are enhanced, thereby realizing the coexistence of two kinds of lights. Further, the optical fiber module is configured for outputting a regulation light or an imaging light, in order to achieve the positioning of target cells, the imaging module is configured for receiving, via the optical fiber module, a fluorescence signal emitted by the target area after being illuminated by the imaging light, and determining the detection image corresponding to the target area based on the fluorescence signal. The control module is configured for determining a target regulation area where the target cell is located based on the detection image, so that the target cells can be precisely regulated in the target regulation area via the regulation light.

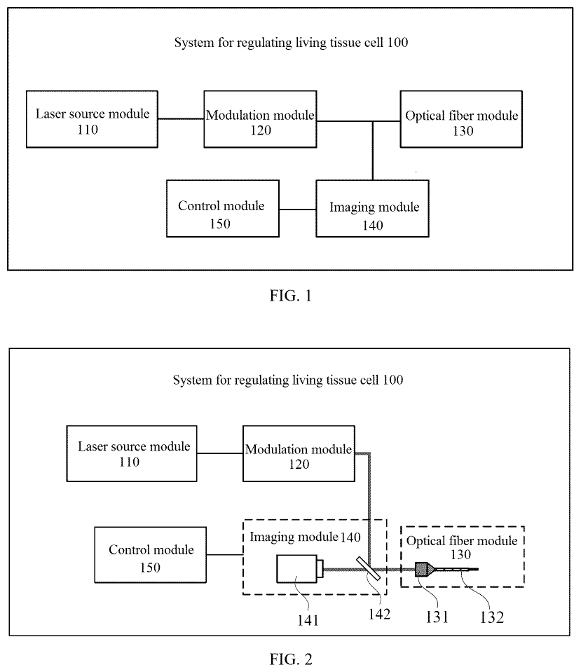

The system for regulating a living tissue cell provided by the embodiments of the present application will be described in detail below with reference to FIGS. 1-8.

FIG. 1 is a schematic structural diagram of a system for regulating a living tissue cell provided by an embodiment of the present application. As shown in FIG. 1, the system 100 for regulating living tissue cells comprises: a laser source module 110, a modulation module 120, an optical fiber module 130, an imaging module 140, and a control module 150.

The laser source module 110 is configured to generating a first laser and a second laser, where the first laser is configured for regulating a target cell based on optogenetics, and the second laser is configured for imaging the target area.

The first laser and the second laser are emitted by different laser devices. It should be understood that the wavelengths of the first laser and the second laser are different. The wavelength of the first laser depends on the target cell to be modulated. The second laser has a wavelength different from the wavelength of the first laser and can be configured for imaging a target area.

The target area refers to the area to be regulated in the living tissue where the target cell is located. It should be understood that the area to be regulated in the living tissue may be located in a superficial area of the living tissue, or may be located a deep area of the living tissue. Although the application scenarios in the embodiments of the present application are mainly aimed at imaging, target cell positioning, and regulation in the deep area of the living tissue, it is also applicable to imaging, target cell positioning, and regulation in the superficial area of the living tissue.

For example, for the brain, the area to be regulated can be a superficial area of the brain or a deep area of the brain.

The first laser and the second laser generated in the laser source module 110 are injected into the modulation module 120 to realize the modulation of the first laser and the second laser.

The modulation module 120 is configured to modulate the received first laser based on a preset modulation strategy to determine the regulation light, and modulate the received second laser based on the preset modulation strategy to determine the imaging light.

The preset modulation strategy refers to a strategy for wavefront modulation of the first laser and the second laser, and the wavefront modulation of the first laser and the second laser is respectively achieved through different strategies in the preset modulation strategy.

In some embodiments, the preset modulation strategy comprises a first modulation strategy and a second modulation strategy. The first modulation strategy is configured for performing the wavefront modulation on the first laser. The second modulation strategy is configured for performing the wavefront modulation on the second laser.

In this case, during the wavefront modulation of the first laser based on the first modulation strategy, the second laser can also be modulated based on the first modulation strategy. Similarly, during the wavefront modulation of the second laser based on the second modulation strategy, the first laser can also be modulated based on the second modulation strategy. However, both the result of modulating the second laser based on the first modulation strategy and the result of modulating the first laser based on the second modulation strategy are not required in the imaging or control process. Therefore, in the modulation module 120, a screening unit can be provided. By adopting the screening unit, the first laser and the second laser modulated based on the first modulation strategy are screened to determine a first laser after modulation (i.e., the regulation light) as an output light of the modulation module 120 in such condition, or alternatively, by adopting the screening unit, the first laser and the second laser modulated based on the second modulation strategy are screened to determine a second laser after modulation (i.e., the imaging light) as an output light of the modulation module 120 in such condition.

The output light of the modulation module 120, comprising the regulation light and the imaging light, is output through the optical fiber module 130. That is to say, the optical fiber module 130 is configured for outputting the regulation light or the imaging light.

It should be understood that for the modulated output light, the regulation light is like the first laser, both the two being configured to control the target cell; the imaging light is like the second laser, both the two being used for imaging the target area.

It should be understood that both the regulation light and the imaging light are output through the optical fiber module 130. The optical fiber in the optical fiber module is a multimode optical fiber. The multimode optical fiber can transmit lights of multiple modes at the same time. In the embodiments of the present application, the multimode optical fiber is configured to transmitting the regulation light and the imaging light.

A core diameter of the multimode optical fiber (usually 50 microns to 100 microns) can provide higher light flux and greater flexibility through the multimode optical fiber. Moreover, the small core diameter of multimode optical fiber can reduce damage to the living tissue during the use of the system. The system with small damage to living tissue can expand the application of the system in various implementation scenarios.

In an embodiment of the present application, the regulation light and the imaging light are integrated into a single optical fiber module 130, and living tissue is imaged and regulated through the single optical fiber module, thereby simplifying the design of the system.

In some embodiments, the optical fiber module 130 comprises a fiber collimator (FC) and a multimode optical fiber (MMF). The regulation light and the imaging light are focused into the multimode optical fiber through the fiber collimator, emitted from an output end of the multimode optical fiber, and then emitted into the target area.

In order to precisely emitting the regulated light into an expected deep target point in the living tissue, the visualization of the living tissue and determination of the insertion path of the optical fiber module should be adopted, therefore, it is necessary to realize the visualization of the living tissue through the imaging module 140.

The imaging module 140 is configured for receiving a fluorescence signal in the target area through the optical fiber module and determining a detection image corresponding to the target area based on the fluorescence signal, where the fluorescence signal is a light emitted by the target area after being illuminated by the imaging light

It should be understood that the imaging light is output through an output end of the optical fiber module 130 and converge to form a point. If the detection image corresponding to the target area is determined, the imaging light output through the output end of the optical fiber module 130 needs to be changed so that the point where the output converging point can scan an entire field of view corresponding to the target area, thereby enabling synthesis of the detection image corresponding to the target area.

That is to say, the imaging light outputs a light field at the output end of the fiber optic module 130. By changing the preset modulation strategy in the modulation module, the light field entering the fiber optic module 130 can be regulated, thereby concentrating the energy at the output end of the fiber optic module 130 into points one by one, and then the entire field of view corresponding to the target area is scanned. The fluorescence generated in the target area is received by the optical fiber module and reversely transmitted to the imaging module 140. According to the intensity of the feedback fluorescence signal corresponding to each scanning point, the detection image of the target area corresponding to the entire field of view can be reconstructed.

In some embodiments, the reflected fluorescence signal is filtered and separated by a dichroic mirror, and then collected by a photodetector in the imaging module 140. After collecting the reflected fluorescence signal, the photodetector calculates the intensity of the energy and synthesizes the detection image corresponding to the target area.

The photodetector is based on the photoelectric effect. When light is emitted to the photosensitive surface of the photodetector, photoelectrons or excited electrons are generated. These electrons then generate electrical signals inside the photodetector. That is, the photodetector is configured to detect and convert light signals into electrical signals. The photodetector may be a photomultiplier tube (PMT).

The control module 150 is in communication connection with the imaging module 140. The control module 150 is configured for determining a target regulation area where the target cell is located based on the detection image, in which, the target regulation area belongs to the area to be regulated.

In some embodiments, the control module is further in communication connection with the modulation module, and the control module is further configured for determining a preset modulation strategy. For example, the preset modulation strategy comprises a first modulation strategy and a second modulation strategy. The first modulation strategy is configured for wavefront modulation of the first laser; and the second modulation strategy is configured for wavefront modulation of the second laser.

FIG. 2 is a schematic structural diagram of a system for regulating a living tissue cell provided by an embodiment of the present application. As shown in FIG. 2, the optical fiber module 130 comprises an optical fiber collimator 131 and a multimode optical fiber 132. The imaging module 140 comprises a photodetector 141 and a first dichroic mirror 142.

It should be understood that an outside of the multimode optical fiber 132 may be provided with a protective structure, such as a protective jacket, etc. In some embodiments, the protective structure on the outside of the multimode optical fiber 132 may be arranged in zones, for example, a diameter of the multimode optical fiber 131 at a side thereof close to the optical fiber collimator is larger than the diameter of the multimode optical fiber of at another side thereof close to the living tissue, so that the optical fiber module 130 is easy to be grasped during operation and can reduce the damage when entering the living tissue.

In some embodiments, the imaging module 140 also comprises a filter. There may be interference light in the fluorescence generated in the target area. The filter filters out light of other colors and only retains the fluorescence band, thereby improving the precision in reconstruction of the detection image by the imaging module.

It should be understood that in the light path of the entire system, in order to reduce hardware interference between different devices, a reflector can be provided to change the direction of the light path. The change in the direction of the light path does not affect the realization of the functions of different devices. Therefore, in the embodiments of the present application, the arrangement of the reflector is not limited herein. In practical applications, it can be provided based on the actual scene.

In some embodiments, the modulation module comprises a light regulation unit and a modulation unit. The light regulation unit is configured for expanding or scaling a process light passing through the modulation module. The process light at least comprises the first laser and the second laser. The modulation unit is configured for adjusting a phase and an intensity of the process light based on the preset modulation strategy.

It should be understood that in the modulation module, the first laser and the second laser are adjusted through the light regulation unit and the modulation unit. The process light comprises forms of the first laser and the second laser in various stages in the modulation module. For example, the light after wavefront adjustment by the modulation unit also belongs to the process light.

In some embodiments, the preset modulation strategy can achieve wavefront shaping (adjustment of phase and intensity) and light field adjustment in only one way.

In some embodiments, the light regulation unit comprises: a first lens assembly and a second lens assembly. The modulation unit comprises a holographic modulation assembly. The first lens assembly is configured for expanding the first laser and the second laser. The holographic modulation assembly is configured for adjusting a phase and an intensity of the first laser and the second laser after being expanded based on a preset modulation strategy to obtain a first output light and a second output light. The second lens assembly is configured for scaling the first output light and the second output light and determining the regulation light and the imaging light.

The preset modulation strategy realizes the adjustment of the expanded first laser and the expanded second laser through the preset hologram. The holographic modulation assembly realizes wavefront adjustment of the first laser and the second laser by displaying a preset hologram.

The above-mentioned combination of the first lens assembly, the holographic modulation assembly, and the second lens assembly realizes the wavefront adjustment of the first laser and the second laser, in which, the first laser and the second laser pass through the first lens assembly, the holographic modulation assembly, and the second lens assembly, respectively, so as to determine the regulation light configured for regulating the target cell and the imaging light configured for imaging the target area, so that the regulation light and the imaging light can be output through the same optical fiber module, thereby making the system can not only image the target area and precisely locate the target cell, but also control the target cell.

In some embodiments, the light regulation unit further comprises a screening assembly. The screening assembly is configured for screening a first target order of the first output light from different diffraction orders corresponding to the first output light and a first target order of the second output light from different diffraction orders corresponding to the second output light. The regulation light is a light whose diffraction order is the first target order in the first output light, and the imaging light is a light whose diffraction order is the first target order in the second output light.

It should be understood that the wavelengths of the first laser and the second laser are different, and by adjusting output light fields of the expanded first laser and the expanded second laser based on the preset hologram, the determined first output light and second output light are also different. Based on the preset hologram, both the first output light and the second output light can have corresponding target orders that can pass through the screening assembly, so that the determined regulation light and imaging light can be output through the same optical fiber module, thereby simplifying the part inserted into the living tissue.

For example, both the first lens assembly and the second lens assembly can be realized through a 4F system, and the holographic modulation assembly can be realized through a spatial light modulator (SLM), such as a high-speed digital micromirror device (DMD), etc. The modulation module realizes wavefront modulation through the preset modulation strategy, and performs high-speed and diffraction-limited focusing through the optical fibers to achieve precise positioning of target cell.

The 4F system is composed of two lenses with different focal lengths; the structure of a high-speed digital micromirror device usually comprises an array composed of many tiny mirrors. These mirrors can be independently controlled through electrostatic attraction or other means to achieve light modulation.

To display the pattern of a preset hologram on the DMD, the digital information of the preset hologram needs to be loaded into the DMD, and the display of the preset hologram is achieved by controlling the deflection of the micromirrors in the DMD.

In some embodiments, the control module is further in communication connection with the modulation module. The control module is further configured for determining the preset modulation strategy, that is, the control module determines the pattern of the preset hologram and controls the DMD to display the pattern of the preset hologram.

It should be understood that the wavelengths of the first laser and the second laser are different, so there are also differences in the wavefront modulation of the first laser and the second laser based on the preset modulation strategy. Therefore, the preset hologram used for the first laser modulation is different from the preset hologram used for the second laser modulation.

In some embodiments, the preset modulation strategy comprises a first modulation strategy and a second modulation strategy. The first modulation strategy is configured for wavefront modulation of the first laser; and the second modulation strategy is configured for wavefront modulation of the second laser. The first modulation strategy comprises a first preset hologram, and the second modulation strategy comprises a second preset hologram. Both the first preset hologram and the second preset hologram belong to the preset hologram.

In some embodiments, the screening assembly comprises a small hole element, and the second lens assembly is provided with a small hole element, and the small hole element is configured to screening the first target order of the first output light from different diffraction orders corresponding to the first output light and a first target order of the second output light from different diffraction orders corresponding to the second output light. The regulation light is a light whose diffraction order is the first target order in the first output light, and the imaging light is a light whose diffraction order is the first target order in the second output light.

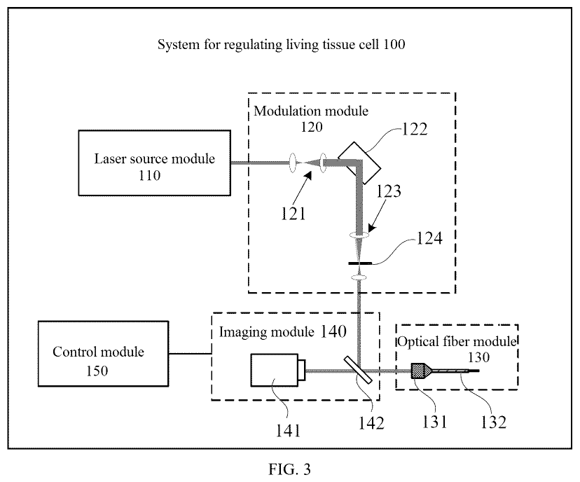

FIG. 3 is a schematic structural diagram of a system for regulating a living tissue cell provided by an embodiment of the present application. As shown in FIG. 3, the modulation module 120 comprises a first 4F system 121, a high-speed digital micromirror device (DMD) 122, a second 4F system 123, and a small hole element 124, in which, the small hole element 124 is arranged in the second 4F system 123 and is configured to block the light whose diffraction order is not the first target order.

It should be understood that the first laser and the second laser emitted by the laser source module enters the modulation module 120 of the first 4F system through the same optical path, and the first laser and the second laser are expanded through the first 4F system 121 to cover the entire DMD 122, and then the preset hologram pattern is displayed on the DMD 122, in this way, the modulation (comprising the phase and the intensity, etc.) of the expanded first laser and expanded second laser is realized, thereby achieving the wavefront shaping.

The first laser and the second laser are modulated simultaneously through the same preset hologram, and the first laser and the second laser have different wavelengths and have different diffraction angles. Therefore, the first laser and the second laser pass through DMD 122, then the first output light and the second output light are output correspondingly.

If the preset hologram at this time is configured to control the first laser, the corresponding small hole element can pass the light whose diffraction order is the first target order in the first output light, and the lights of all diffraction orders in the second output light cannot pass through the small hole element.

If the preset hologram at this time is configured to control the second laser, the corresponding small hole element can pass the light whose diffraction order is the first target order in the second output light, and the lights of all diffraction orders in the first output light cannot pass through the small hole element.

That is to say, through the small hole element, it is possible to control whether the light output by the modulation module 120 is the imaging light or the regulation light. The regulation light is the light whose diffraction order is the first target order in the first output light, and the imaging light whose diffraction order is the first target order in the second output light.

It should be understood that the refresh rate of the DMD 122 is very high, so the raster scanning and switching speed of the two laser sources are very fast, so that the light output through the multimode optical fiber 132 of the optical fiber module 130 contains the imaging light and the regulation light, and due to the DMD 122 switches quickly, the imaging light and the regulation light do not interfere with each other and output precisely.

The above-mentioned wavefront shaping achieved through DMD 122 can enable the light to achieve high-speed, diffraction-limited focusing and precise positioning, and meanwhile, can pre-compensate the modal dispersion of light, overcome the limitations of transmission of low spatial and temporal resolution stimulation, and measure the complex light transfer function in the multimode optical fiber 132, and can form a diffraction-limited focus of any light at a distal end of the multimode optical fiber 132, whereby achieving high-precision light regulation (stimulation, inhibition, etc.), that is, single cell regulation as well as real-time imaging of the imaging light with high-resolution raster scanning can be realized.

The imaging module 140 comprises a photodetector 141 and a first dichroic mirror 142. The fluorescence emitted by the target area after being illuminated by the imaging light is again received by the multimode optical fiber 132 and is reversely transmitted to the imaging module 140. The reversely transmitted light passes through the first dichroic mirror 142, and a reflected fluorescence signal is filtered out and is then collected by the photodetector 141. After collecting the reflected fluorescence signal, the photodetector 141 calculates the intensity of the energy and synthesizes a detection image corresponding to the target area.

After being output from the output end of the multimode optical fiber, the imaging light converges to a point in the field of view of the target area, and then scans the field of view of the entire target area. According to the intensity of the fluorescence signal fed back at each corresponding scanning point, the fluorescence imaging of the entire field of view is reconstructed, that is, the detection image is determined.

The scanning of the field of view of the target area may be from left to right or from top to bottom.

If the obtained detection image contains target cell, the target cell can be located and the corresponding position of the target cell, that is, the target regulation area, can be determined.

Through the imaging module, images of areas to be regulated in living tissues can be fed back, target cells can be precisely located, and real-time feedback on the regulation effects of target cells can be provided.

In some embodiments, the laser source module comprises: a first light source unit, a second light source unit, and a beam splitting unit. The first light source unit is configured for emitting the first laser, the second light source unit is configured to emitting the second laser, and the beam splitting unit is configured for dividing the second laser into a second signal beam and a reference beam. The second signal beam is configured for imaging the target area, and the reference beam is configured for calibrating the second signal beam. Both the first laser unit and the second laser unit may be light sources that can emit beams of fixed wavelengths. The beam splitting unit may be a component such as a beam splitter configured for splitting light.

It should be understood that the second signal beam is calibrated by the reference beam to generate the imaging light. The reference beam interferes with the imaging light that passes through the fiber optic module, thereby precisely measuring the transmission matrix of the imaging light passing through the fiber optic module.

In some embodiments, the beam splitting unit is further configured for dividing the first laser into a first signal beam, and the first signal beam is configured for regulating the target cell. The beam splitting unit may comprise two beam splitters, one of which is configured for splitting the first laser and the other one is configured for splitting the second laser.

In some embodiments, the laser source module comprises: a first light regulation unit, a second light regulation unit, and a combining unit. The first light regulation unit is configured for controlling an intensity of the first laser. The second light regulation unit is configured for controlling an intensity of the second laser. The combining unit is configured for combining the first laser and the second laser into one channel of light, wherein the first laser passes through the combining unit, and the second laser is reflected by the combining unit. The first light regulation unit and the second light regulation unit can be one element or a combination of multiple elements, for example, can be a half-wave plate, a variable attenuator, etc., or a half-wave plate and a polarizer, or a combination of a half-wave plate and a bias variable attenuator, and the combining unit can be realized through a dichroic mirror, a lens, etc.

It should be understood that a dichroic mirror can separate light of different wavelengths, and similarly, two lights with different wavelengths can also be combined into one beam through a dichroic mirror.

However, the light emitted by multimode optical fiber spreads out, producing a wide illumination area or random speckle pattern not far from the end of the optical fiber. Therefore, a calibration module is needed to improve the precision of the light emitted by multimode optical fiber.

In order to achieve calibration of the output light of the optical fiber module, the system 100 further comprises a calibration module 160, and the calibration module 160 comprises a reference unit and a recording unit. The reference unit is configured for converging the reference beam to the recording unit. The recording unit is configured for: acquiring a combined beam, and determining a first transmission matrix based on interference between different beams in the combined beam. The combined beam comprises the reference beam and an object beam, and the object beam refers to the regulation light or the imaging light which is irradiated on the target area.

In some embodiments, the reference unit further comprises a shutter assembly. The shutter assembly is configured for controlling the reference beam to converge to the recording unit or blocking the reference beam from converging to the recording unit. In a case where the shutter assembly blocks the reference beam from converging to the recording unit, the recording unit is configured for acquiring the object beam and determining a second transmission matrix based on the object beam.

It should be understood that the first light source unit and the second light source unit are respectively calibrated by whether to consider the reference beam of the second laser, and when calibrating the first light source unit, the object beam is the regulation light that illuminates to the target area, and when calibrating the second light source unit, the object beam is the imaging light that illuminates the target area.

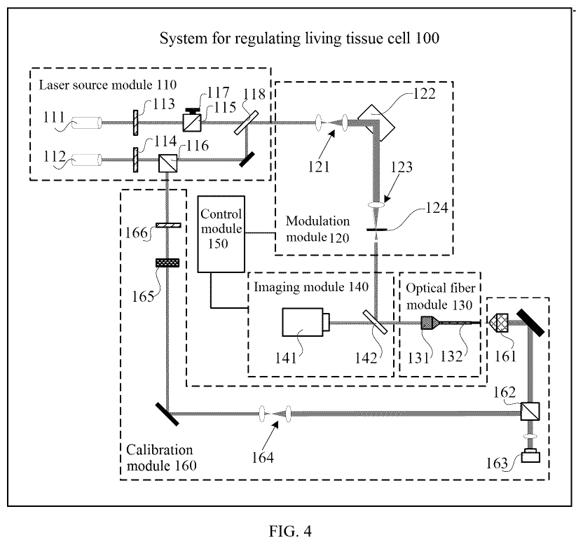

FIG. 4 is a schematic structural diagram of a system for regulating a living tissue cell provided by an embodiment of the present application. As shown in FIG. 4, the laser source module 110 comprises: a first light source 111, a second light source 112, a first half-wave plate 113, a second half-wave plate 114, a first polarization beam splitter115, a second polarization beam splitter116, and a second dichroic mirror 118.

The first laser is emitted from the first light source 111, has an intensity of the first laser controlled when passing through the first half-wave plate 113 and the first polarization beam splitter 115, and is then emitted to the second dichroic mirror 118. The second laser is emitted from the second light source 112, has an intensity of the second laser controlled when passing through the second half-wave plate 114 and the second polarization beam splitter 116, and is then divided into a second signal beam and a reference beam when passing through the second polarization beam splitter 116. The second signal beam is emitted to the second dichroic mirror 118, which further emits the first laser and the second signal light to the modulation module.

In some embodiments, the laser source module 110 further comprises a beam blocker 117 disposed on the first polarization beam splitter 115, by which, the first signal beam is separated from the first laser. The beam separated by the first polarization beam splitter 115 from the first laser can also be blocked by the beam blocker 117. In this way, the precision of the beam in the first laser entering the modulation module is improved.

In such condition, the first laser is emitted from the first light source 111, has the intensity of the first laser controlled when passing through the first half-wave plate 113 and the first polarization beam splitter 115, in which, when passing the first polarization beam splitter 115, the first signal beam can be separated from the first laser and then emitted to the second dichroic mirror 118. The second laser is emitted from the second light source 112, has the intensity of the second laser controlled when passing through the second half-wave plate 114 and the second polarization beam splitter 116, and is then divided into a second signal beam and a reference beam when passing through the second polarization beam splitter 116. The second signal beam is emitted to the second dichroic mirror 118, which further emits the first laser and the second signal light to the modulation module.

For descriptions of the modulation module 120, the optical fiber module 130, and the imaging module 140, reference may be made to the corresponding descriptions in the corresponding embodiment of FIG. 3.

It should be understood that for the preset hologram displayed by the DMD 122 in the modulation module 120, the display of the DMD 122 can be controlled by the control module, and based on the modulation of different lasers, different preset holograms are displayed correspondingly.

In some embodiments, the preset hologram is a Lee hologram, that is, through the DMD 122, the second 4F system 123, and the small hole element 124, the DMD 122 outputs the first output light and the second output light through the preset hologram, and the corresponding regulation light and imaging light are determined and output through the second 4F system 123 and the small hole element 124.

As shown in FIG. 4, the calibration module 160 comprises: a recording unit, which is composed of an objective lens 161, a third polarization beam splitter 162, and a camera 163, and a reference unit, which is composed of a third 4F system 164, a shutter 165, and a third half-wave plate 166.

The calibration process comprises: calibration of the first light source 111 and calibration of the second light source 112.

The process of calibration of the first light source 111 comprises: the first signal beam separated by the first polarization beam splitter 115 from the first laser is emitted to the target area through the modulation module 120 and the optical fiber module 130 and is referred to the object beam. The object beam is collected by the objective lens and then recorded by the camera 163, and a first transmission matrix of the multimode optical fiber is calculated. It should be understood that when the first light source 111 is calibrated, the shutter 165 is in a closed state and the camera 163 only records object beam.

The process of calibration of the second light source 112 comprises: the reference beam separated by the second polarization beam splitter 116 from the second laser passes through the third half-wave plate 166, the shutter 165, and the third 4F system 164, respectively, then is combined at the third polarization beam splitter 162 with the object beam passing through the objective lens, and then recorded by the camera 163, and a second transmission matrix of the multimode optical fiber is calculated. In such condition, the object beam is a light illuminated on the target area after the second signal is separated by the second polarization beam splitter 116 from the second laser and passes through the modulation module 120 and the optical fiber module 130.

The above calibration process is performed before the imaging and regulation of the system.

FIG. 5 is a schematic structural diagram of a system for regulating a living tissue cell provided by an embodiment of the present application. As shown in FIG. 5, a schematic diagram of a scene in which the system for regulating a living tissue cell is applied to a mouse brain sample. In the light path of the entire system, in order to reduce hardware interference between different devices, a reflector can be provided to change the direction of the light path. The change in the direction of the light path does not affect the realization of the functions of different devices. Therefore, in the embodiments of the present application, the arrangement of the reflector is not limited herein. In practical applications, it can be provided based on the actual scene.

When applied in this scenario, the system can refer to the corresponding description in the corresponding embodiment of FIG. 4.

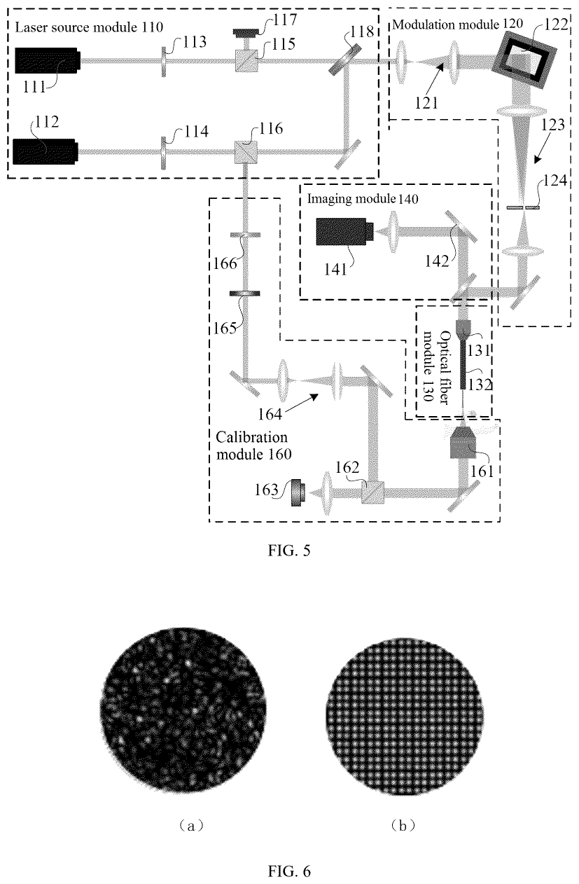

FIG. 6 is a comparison diagram showing effects of wavefront shaping provided by the embodiment of the present application, applied to the system for regulating the living tissue cell shown in FIG. 5. As shown in FIG. 6, part (a) in FIG. 6 shows no effects of the wavefront shaping. Part (b) of FIG. 6 shows the effect after performing the wavefront shaping. By measuring and pre-compensating the complex optical transfer function of the multimode optical fiber through the wavefront shaping, a diffraction-limited focus of any light can be formed at the distal end of the multimode optical fiber, which can realize high-precision light modulation and high-resolution real-time imaging of the raster scanning.

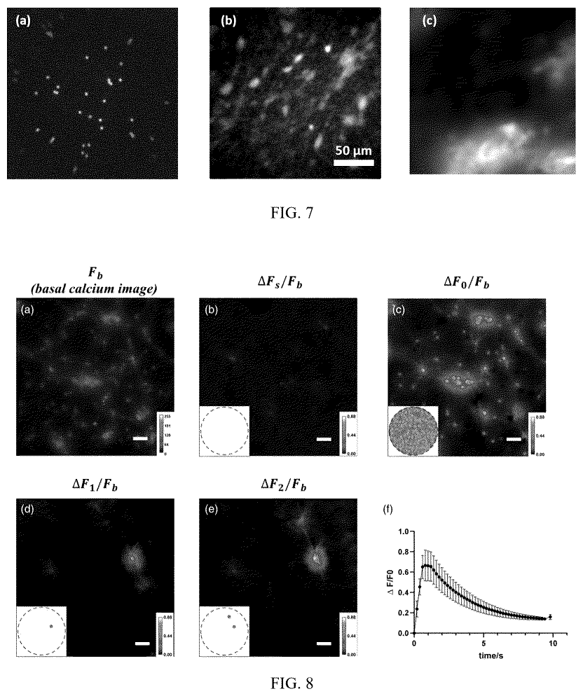

FIG. 7 is imaging results of a system for regulating a living tissue cell provided by an embodiment of the present application. As shown in FIG. 7, part (a) of FIG. 7 shows the fluorescence imaging of fluorescent beads, and part (b) of FIG. 7 shows the fluorescence imaging of target cells in a mouse brain slice, and part (c) in FIG. 7 is fluorescence imaging of target cells in a mouse brain.

It should be understood that the imaging process is performed after calibration.

FIG. 8 regulation results of a system for regulating a living tissue cell provided by an embodiment of the present application. As shown in FIG. 8, part (a) of FIG. 8 is calcium imaging under blue light illumination, as the background F_{b}; part (b) of FIG. 8 shows neurons under a quiet condition without light stimulation, recorded as a difference between the image and the background ΔFₛ/F_{b}; and part (c) of FIG. 8 shows that there is no wavefront shaping, and the light output from the multimode optical fiber is a random spot pattern, recorded as a difference between the image and the background ΔF₀/F_{b}, and confirming that all neurons in the field of view appear to be activated. Part (d) and part (e) of FIG. 8 are based on the obtained projection matrix, the phase masks configured to generating one and two focus areas can be calculated and uploaded to the DMD. The corresponding differences of the image relative to the background image are shown in part (d) and part (e) of FIG. 8, neuron-selective activation is demonstrated for precise neuronal activation through multimode optical fiber. Part (f) of FIG. 8 shows the statistical data of repeated stimulation of the target cells in part (d) of FIG. 8.

After the target regulation area where the target cell is located is determined by imaging, the target cells can be stimulated and regulated.

It should be understood that the above are examples of application scenarios and do not limit the application scenarios of the present application in any way.

It should be understood that the above examples are to help those skilled in the art understand the embodiments of the present application, but are not intended to limit the embodiments of the present application to the specific numerical values or specific scenarios illustrated. Those skilled in the art can obviously make various equivalent modifications or changes based on the above embodiments, and such modifications or changes also fall within the scope of the embodiments of the present application.

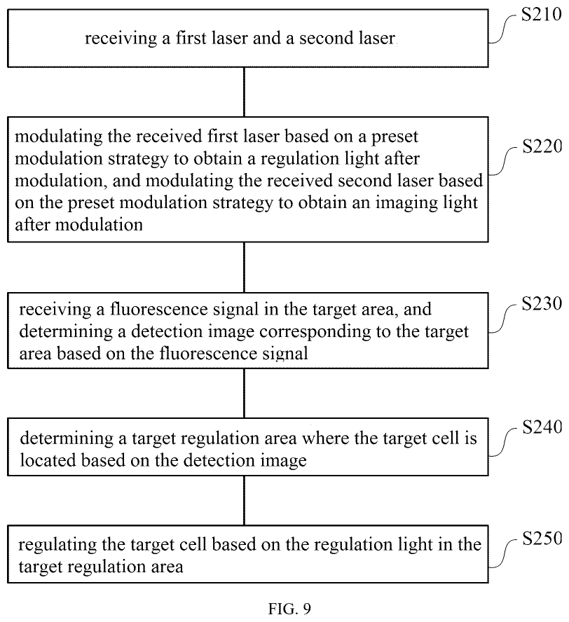

The system for regulating a living tissue cell according to the embodiments of the present application is described in detail above with reference to FIGS. 1-8. Hereinbelow, a method for regulating a living tissue cell according to the present application will be described in detail with reference to FIG. 9. It should be understood that the method for regulating the living tissue cell in the embodiments of the present application are applied to the system for regulating the living tissue cell, that is, for the implementation process of the following method embodiments, reference can be made to the corresponding descriptions in the foregoing system embodiments.

FIG. 9 is a schematic flow chart of a method for regulating a living tissue cell provided by an embodiment of the present application. As shown in FIG. 9, the method comprises:
S210. receiving a first laser and a second laser;
in which, the first laser is configured for regulating a target cell based on optogenetics, and the second laser is configured for imaging a target area, and the target area refers to an area to be regulated in a living tissue where the target cell is located;
S220. modulating the received first laser based on a preset modulation strategy to obtain a regulation light after modulation, and modulating the received second laser based on the preset modulation strategy to obtain an imaging light after modulation;
in which, the preset modulation strategy refers to a strategy for wavefront modulation of the first laser and the second laser;
S230: receiving a fluorescence signal in the target area, and determining a detection image corresponding to the target area based on the fluorescence signal;
in which, the fluorescence signal is a light emitted by the target area after being illuminated by the imaging light;
S240. determining a target regulation area where the target cell is located based on the detection image;
in which, the target regulation area belongs to the area to be regulated; and
S250. regulating the target cell based on the regulation light in the target regulation area.

Referring to the corresponding descriptions in the foregoing system embodiments, the method for regulating a living tissue cell comprises: calibration, imaging, and regulation. This method provides a view of the target area when inserting a multimode optical fiber, and can more precisely place the stimulation source in the target area where the target cell is located, so as to achieve the regulation. The view of the regulation process can also be displayed in real time. Such real-time feedback can reliably target deep regions of living cells (e.g., deep brain regions) with high precision.

Embodiments of the present application further provide a computer-readable storage medium storing a computer program, which, when being executed by a computer, causes the method for regulating a living tissue cell according to any of the method embodiments of the present application to be performed. The computer program may be a high-level language program or an executable object program.

Embodiments of the present application further provide a computer program product, which, when running on a mobile terminal, causes the mobile terminal to implement the method for regulating the living tissue cell according to any method embodiment of the present application.

In the above embodiments, each embodiment is described with its own emphasis. For parts that are not detailed or recorded in a certain embodiment, the relevant descriptions of other embodiments may be referred to.

Those of ordinary skill in the art will appreciate that the units and algorithm steps of each example described in conjunction with the embodiments disclosed herein can be implemented with electronic hardware, or a combination of computer software and electronic hardware. Whether these functions are performed in hardware or software depends on the specific application and design constraints of the technical solution. Skilled artisans may implement the described functionality using different methods for each specific application, but such implementations should not be considered beyond the scope of the present application.

Those skilled in the art can clearly understand that for the convenience and simplicity of description, the specific working processes of the systems, devices, and units described above can be referred to the corresponding processes in the foregoing method embodiments, and will not be described again here.

In the several embodiments provided in the present application, it should be understood that the disclosed devices and methods can be implemented in other ways. For example, the device embodiments described above are only illustrative; for example, the division of units is only a logical function division, and there may be other division methods in actual implementation; for example, multiple units or components may be combined or integrated into another system, or some features can be ignored, or not implemented. On the other hand, the coupling or direct coupling or communication connection between each other shown or discussed may be through some interfaces, and the indirect coupling or communication connection of the devices or units may be in electrical, mechanical, or other forms.

A unit described as a separate component may or may not be physically separate. A component shown as a unit may or may not be a physical unit, that is, it may be located in one place, or it may be distributed to multiple network units. Some or all of the units can be selected according to actual needs to achieve the purpose of the solution of this embodiment.

In addition, each functional unit in various embodiments of the present application may be integrated into one processing unit, or each unit may exist physically alone, or two or more units may be integrated into one unit.

The above are only specific embodiments of the present application, but the protection scope of the present application is not limited thereto. Any person familiar with the technical field can easily think of changes or replacements within the technical scope disclosed in the present application, and all of them should fall within the protection scope of the present application. Therefore, the protection scope of the present application should be subject to the protection scope of the claims.

1. A system for regulating a living tissue cell, comprising:
a laser source module, configured for generating a first laser and a second laser, wherein the first laser is configured for regulating a target cell based on optogenetics, and the second laser is configured for imaging a target area, and the target area refers to an area to be regulated in a living tissue where the target cell is located;
a modulation module, configured for modulating the received first laser based on a preset modulation strategy to determine a regulation light and modulating the received second laser based on the preset modulation strategy to determine an imaging light, wherein the preset modulation strategy refers to a strategy for wavefront modulation of the first laser and the second laser;
an optical fiber module, configured for outputting the regulation light or the imaging light, wherein the regulation light is configured for regulating the target cell;
an imaging module, configured for receiving a fluorescence signal in the target area through the optical fiber module and determining a detection image corresponding to the target area based on the fluorescence signal, wherein the fluorescence signal is a light emitted by the target area after being illuminated by the imaging light; and
a control module, in communication connection with the imaging module and configured for determining a target regulation area where the target cell is located based on the detection image, wherein the target regulation area belongs to the area to be regulated.

2. The system according to claim 1, wherein the modulation module comprises a light regulation unit and a modulation unit;
the light regulation unit is configured for expanding or scaling a process light passing through the modulation module, wherein the process light at least comprises the first laser and the second laser; and
the modulation unit is configured for adjusting a phase and an intensity of the process light based on the preset modulation strategy.

3. The system according to claim 2, wherein
the light regulation unit comprises: a first lens assembly and a second lens assembly;
the modulation unit comprises a holographic modulation assembly;
the first lens assembly is configured for expanding the first laser and the second laser;
the holographic modulation assembly is configured for adjusting a phase and an intensity of the first laser and the second laser after being expanded based on a preset modulation strategy to obtain a first output light and a second output light; and
the second lens assembly is configured for scaling the first output light and the second output light and determining the regulation light and the imaging light.

4. The system according to claim 3, wherein the light regulation unit further comprises a screening assembly;
the screening assembly is configured for screening a first target order of the first output light from different diffraction orders corresponding to the first output light and a first target order of the second output light from different diffraction orders corresponding to the second output light;
the regulation light is a light whose diffraction order is the first target order in the first output light, and
the imaging light is a light whose diffraction order is the first target order in the second output light.

5. The system according to claim 1, wherein the laser source module comprises a beam splitting unit,
the beam splitting unit is configured for dividing the second laser into a second signal beam and a reference beam;
the second signal beam is configured for imaging the target area; and
the reference beam is configured for calibrating the second signal beam.

6. The system according to claim 5, wherein
the system further comprises a calibration module, and the calibration module comprises a reference unit and a recording unit;
the reference unit is configured for converging the reference beam to the recording unit;
the recording unit is configured for: acquiring a combined beam, and determining a first transmission matrix based on an interference between different beams in the combined beam, wherein the combined beam comprises the reference beam and an object beam, and the object beam refers to the regulation light or the imaging light which is irradiated on the target area.

7. The system according to claim 6, wherein the reference unit further comprises a shutter assembly, the shutter assembly is configured for controlling the reference beam to converge to the recording unit or blocking the reference beam from converging to the recording unit; and
in a case where the shutter assembly blocks the reference beam from converging to the recording unit, the recording unit is configured for acquiring the object beam and determining a second transmission matrix based on the object beam.

8. The system according to claim 1, wherein
the control module is further in communication connection with the modulation module;
the control module is also configured for determining the preset modulation strategy;
the preset modulation strategy comprises a first modulation strategy and a second modulation strategy;
the first modulation strategy is configured for wavefront modulation of the first laser; and
the second modulation strategy is configured for wavefront modulation of the second laser.

9. The system according to claim 1, wherein the laser source module comprises: a first light regulation unit, a second light regulation unit, and a combining unit; wherein
the first light regulation unit is configured for controlling an intensity of the first laser;
the second light regulation unit is configured for controlling an intensity of the second laser;
the combining unit is configured for combining the first laser and the second laser into one channel of light, wherein the first laser passes through the combining unit, and the second laser is reflected by the combining unit.

10. A method for regulating a living tissue cell, applied to a system for regulating a living tissue cell and comprising:
receiving a first laser and a second laser, wherein the first laser is configured for regulating a target cell based on optogenetics, and the second laser is configured for imaging a target area, and the target area refers to an area to be regulated in a living tissue where the target cell is located;
modulating the received first laser based on a preset modulation strategy to obtain a regulation light after modulation, and modulating the received second laser based on the preset modulation strategy to obtain an imaging light after modulation, wherein the preset modulation strategy refers to a strategy for wavefront modulation of the first laser and the second laser;
receiving a fluorescence signal in the target area, and determining a detection image corresponding to the target area based on the fluorescence signal, wherein the fluorescence signal is a light emitted by the target area after being illuminated by the imaging light;
determining a target regulation area where the target cell is located based on the detection image, wherein the target regulation area belongs to the area to be regulated; and
regulating the target cell based on the regulation light in the target regulation area.

11. A non-transitory computer-readable storage medium, storing computer program codes, which, when being executed by a processor, cause the processor to perform the method for regulating the living tissue cell according to claim 10.
